(19) United States
(12) Patent Application Publication
(10) Pub. No.: US 2002/0151836 A1
(43) Pub. Date: Oct. 17, 2002

(54) Portable aspirating and irrigating apparatus and method

(76) Inventor: Bill E. Burden, Altamonte Springs, FL (US)

Correspondence Address: Allen, Dyer, Doppelt, Milbrath & Gilchrist, P.A., 255 South Orange Avenue, Suite 1401, P.O. Box 3791, Orlando, FL, 32802-3791, US

(21) Appl. No.: 10/062,586

(22) Filed: Jan. 31, 2002

Related U.S. Application Data
(60) Provisional application No. 60/265,699, filed on Feb. 1, 2001.

(51) Int. Cl. A61M 1/00

(52) U.S. Cl. 604/35; 604/152

(57) ABSTRACT

An aspirating and irrigating apparatus and method provide rapid and effective aspirating and irrigating of body parts during medical procedures to such a degree that anesthetizing the patient is no longer necessary for that portion of the procedure. The apparatus is portable and includes a piston driven motor which drives a vacuum pump having an input port for providing a vacuum operating to vacuum levels exceeding approximately twenty-seven inches of mercury and an output port for providing air under pressure operating to pressure levels exceeding approximately one hundred and twenty pounds per square inch for permitting rapid fluid flows.

### CROSS REFERENCE TO RELATED APPLICATIONS

This application incorporates by reference and claims priority to Provisional Application having Serial No. 60/265,699 filed Feb. 1, 2001 for ASPIRATOR APPARATUS AND METHOD, commonly owned with the present invention.

### FIELD OF THE INVENTION

The present invention generally relates to medical suction devices and in particular to a medical device and method for controlled and combined aspiration and irrigation of fluids.

### BACKGROUND OF THE INVENTION

Aspirating and irrigating are well known in the medical arts and have played a major role during surgery and general patient preparation for minor to major operations. Suctioning or aspiration of body fluids in medical procedures is a necessary occurrence and as described in U.S. Pat. No. 4,915,691 to Jones et al. has been accomplished in a myriad of ways. By way of example, and as described in U.S. Pat. No. 6,258,067 to Hill, infections of the ear present ongoing problems to both the patient who suffer and the physicians who seek to treat them, and as a result there is a need for an aspirator that allows for increased control. As part of that control, increased speed would allow the physician to complete the task at hand more quickly.

### SUMMARY OF THE INVENTION

In view of the foregoing background, it is therefore an object of the present invention to provide an apparatus and method for quickly and effectively aspirating and irrigating body parts during medical procedures. It is further an object of the present invention to improve the speed in which irrigation and aspiration has taken place thus far to the degree where anesthetizing the patient is no longer necessary for the irrigation and aspiration portion of the procedure.

These and other objects, features and advantages according to the present invention are presented in a portable apparatus for rapidly irrigating and aspirating living body portions to thus avoid anesthetizing the body. The apparatus comprises a housing sized for ease in manually placing the apparatus at desired locations during use. A piston driven motor drives a vacuum pump, both of which are carried by the housing. The vacuum pump includes an input port for providing a vacuum operating to vacuum levels exceeding approximately twenty-seven inches of mercury. In addition, the vacuum pump includes an output port for providing air therefrom under pressure to pressure levels exceeding approximately one hundred and twenty pounds per square inch. Such vacuum and pressure levels permit rapid fluid flows and thus allow a veterinary physician, by way of example, to perform irrigating and aspirating procedures for an animal without the need to anesthetize the animal, unlike that typically performed with well known aspirators and irrigators. A canister is in fluid flow communication with the input port and an aspirator wand is in fluid flow communication with the canister for depositing fluid therein as a result of the vacuum. Vacuum level control means are operable between the input port and the canister for controlling the vacuum developed at the canister and thus at the aspirator wand for receiving body fluids at a preselected flow rate. In one preferred embodiment, the vacuum level control means comprises a gauge for displaying the vacuum level and a manually actuated valve for controlling the vacuum level.

In one embodiment, the housing carries a ceramic filter in fluid communication between the input port and the vacuum control means for filtering flow from the canister to the input port and preventing debris from entering the vacuum pump. An inline filter is operable with the canister for filtering flow therefrom.

A pressure bottle is in fluid flow communication with the output port. A syringe is also in fluid flow communication with the output port for delivering air under pressure therefrom and further in fluid flow communication with the pressure bottle for delivering air under pressure mixed with fluid from the pressure bottle. Flow control means are operable with the syringe for controlling fluid flow from at least one of the output port and the pressure bottle. Pressure level control means are operable with the output port for controlling the air pressure delivered therefrom, and thus to the syringe and pressure bottle, wherein the pressure level control means comprise a gauge for displaying the pressure level and a manually actuated valve for controlling the pressure level. Optionally, a second pressure valve is operable with the output port for limiting pressure delivered therefrom. A plurality of quick-connect connections are positioned inline between at least one of the output port and pressure control means, the pressure control means and the pressure bottle, the pressure bottle and the syringe, and the pressure control means and the syringe.

### BRIEF DESCRIPTION OF THE DRAWINGS

A preferred embodiment of the present invention and alternate embodiments are herein described with reference to the accompanying drawings in which:

FIG. 1 is a front perspective view of one embodiment of an aspirating and irrigating apparatus in keeping with the teachings of the present invention;

FIG. 2 is a right side perspective view of the embodiment of FIG. 1;

FIG. 3 is a rear perspective view of the embodiment of FIG. 1;

FIG. 4 is a block diagram illustrating aspiration and irrigation flow for elements of the embodiment of FIG. 1;

FIG. 4A is a block diagram illustrating an alternate embodiment for the aspiration and irrigation flow elements of FIG. 4; and

FIG. 5 is a partial top view of the embodiment of FIG. 1 illustrating connector quick-release features.

### DETAILED DESCRIPTION OF PREFERRED EMBODIMENTS

The present invention will now be described more fully with reference to the accompanying drawings in which preferred embodiments of the invention are shown and described. It is to be understood that the invention may be embodied in many different forms and should not be construed as limited to the illustrated embodiments set forth herein. Rather, the applicant provides these embodiments so that this disclosure will be thorough and complete, and will convey the scope of the invention to those skilled in the art. Like numbers refer to like elements there through.

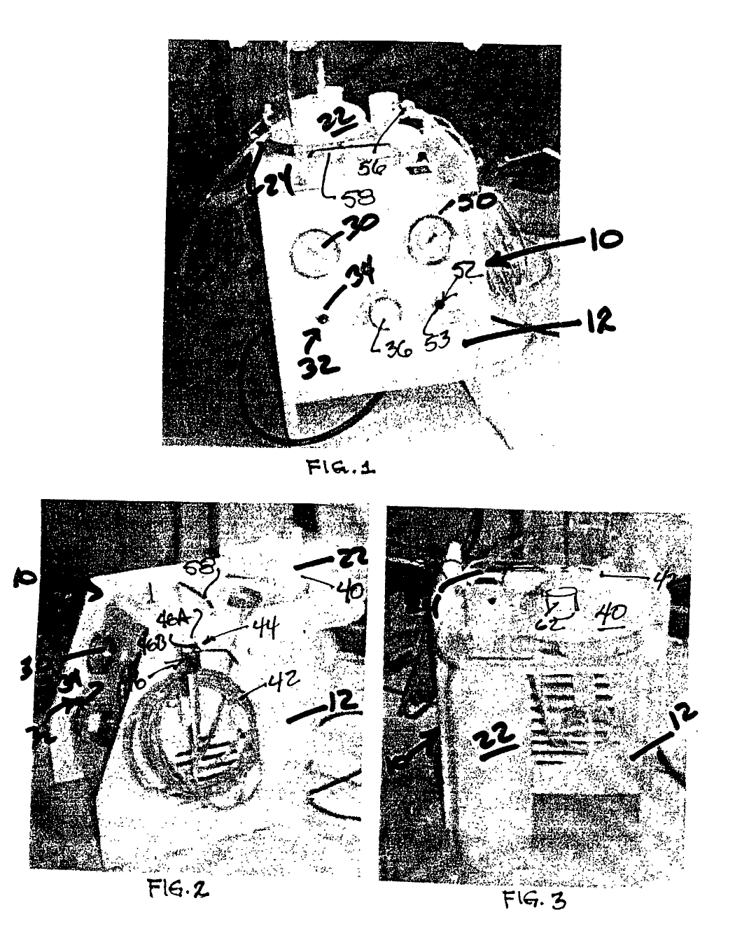

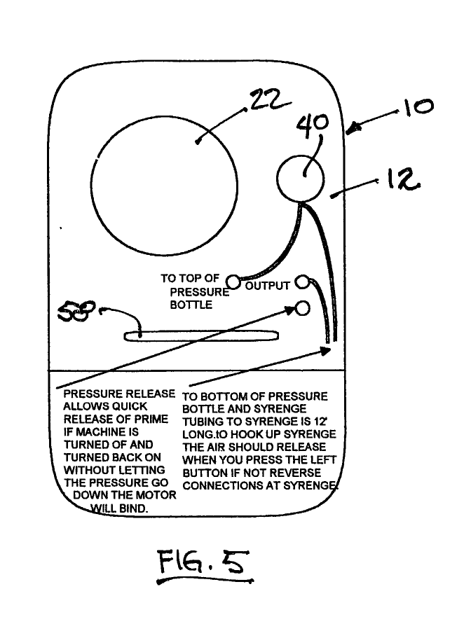

As illustrated initially with reference to FIGS. 1-3, one preferred embodiment of the present invention is herein described, by way of example, as an apparatus 10 comprising a housing 12 sized for ease in manually placing the apparatus at desired locations during use. As illustrated with reference to FIGS. 4 and 4A, a piston driven motor 14 drives a vacuum pump 16, both of which are carried within the housing 12. The vacuum pump 16 includes an input port 18 for providing a vacuum operable to vacuum levels exceeding approximately 27″ of mercury. In addition, the vacuum pump 16 includes an output port 20 for providing air under pressure and operating to pressure levels exceeding approximately 120 psi. Such vacuum and pressure levels permit rapid fluid flows and thus allow a veterinary physician, by way of example, to perform irrigating and aspirating procedures for an animal without the need to anesthetize the animal, unlike that typically performed with well known aspirators and irrigators.

With continued reference to FIGS. 1-4, a canister 22 is connected to the input port 18 using flexible plastic tubing 24. An aspirator wand 26 is connected to the canister 22 using similar tubing 24 for depositing fluid into the canister through use of the vacuum developed by the vacuum pump 16. A vacuum level controller 28 is connected between the input port 18 and the canister 22 for controlling the vacuum developed at the canister and thus at the aspirator wand 26 for receiving body fluids at a preselected flow rate. In one preferred embodiment, the vacuum level controller 28 comprises a vacuum gauge 30 for displaying the vacuum level and a manually actuated valve 32 for controlling the vacuum level to those desired by the physician. The gauge 30 is carried on the front face of the housing 12. A knob 34 is conveniently positioned below the gauge 30 for ease of use by the physician or operator of the apparatus 10.

In one preferred embodiment as herein described by way of example, and as illustrated with reference again to FIGS. 1 and 4, the housing 12 also carries a ceramic filter 36 which is in fluid communication between the input port 18 and the vacuum controller 28 for filtering flow from the canister 22 to the input port and preventing debris from entering the vacuum pump 16. In addition, an inline filter 38 is operable with the canister 22 for filtering flow therefrom.

With reference again to FIGS. 1-4, a pressure bottle 40 is connected to the output port 20 using tubing 42. A syringe 44 is also connected to the output port 20 using similar tubing 44 for delivering air under pressure. In addition, the syringe 44 is connected to the pressure bottle 40 for delivering air under pressure mixed with fluid, such as medications and the like, carried by the pressure bottle. A flow controller 46 is operable with the syringe 44 for controlling fluid flow. In the preferred embodiment herein described by way of example, the controller 46 includes first 46A and second 46B buttons conveniently positioned on the syringe 44 for ease of operation. A pressure level controller 48 is operable with the output port 20 for controlling the air pressure to the syringe 44 and pressure bottle 40. As herein described, the pressure controller 48 comprises a pressure gauge 50 for displaying the pressure level and a manually actuated valve 52 having a knob 53 for controlling the pressure level.

Optionally, a second pressure valve 54 is carried within the housing, out of view for the operator, and operable with the output port 20 for limiting pressure delivered.

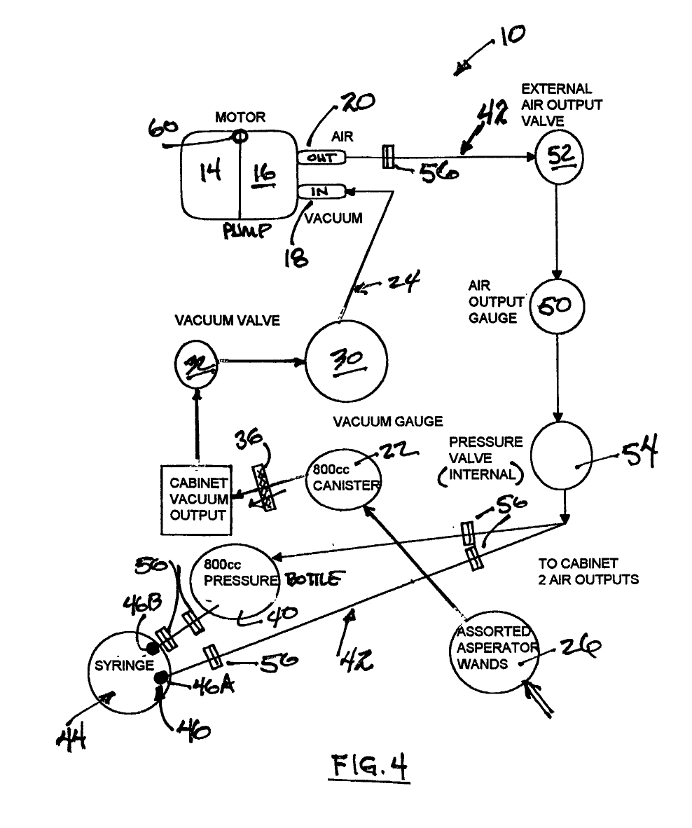

As further described for the preferred embodiment herein described, a plurality of quick-connect connectors 56 are positioned inline between at least one of the output port 20 and pressure controller 48, the pressure controller and the pressure bottle 40, the pressure bottle and the syringe 44, and the pressure controller and the syringe. As illustrated with reference again to FIGS. 4 and 4A, a total of six connectors 56 are operable in the apparatus 10. As illustrated with reference again to FIGS. 1 and 2, a handle 58 is provided for use in transporting the portable apparatus 10. As illustrated with reference again to FIG. 4, and now to FIG. 5, the apparatus 10 includes a pressure release valve operated by a button 60 for releasing a pressure prime of the pump 16 when the apparatus is turned off and back on to avoid a binding of the motor 14.

Embodiments of the present invention have been tested and shown to satisfy the needs above addressed. Features of these embodiments include power and control superior to that known in the art provided with multi-functionality, small size and lightweight portability to help the busy veterinary doctor, by way of example, improve performance. The apparatus 10 as above described provides a precise surgical suction up to 27″ Hg where typical units provide less than 20″ Hg. The invention has proven to be successful for flushing ears during ear infection procedures, useful with Lavage body cavities and Lavage wounds, superior for evacuation of the body cavity in cases of Hydrothorax and Hydro peritoneum, and desirable for flushing and vacuuming the sinus cavity. By way of example, small animal sinuses can be flushed with saline or medication and the liquid can be vacuumed before it goes down the throat.

During ear infection treatment, debris can be quickly cleaned, irrigated and washed out with saline and vacuumed. Local testing has shown that most animals don't need sedation due to the gentle, quiet and precise operation of the apparatus 10 which can flush an open cavity during surgery with saline and vacuum it quickly to avoid using gauze pads and swabs, which leave debris. Multiple and distinct wands and probes are expected to be used with the apparatus 10, the wand 26 being described by way of example only. It is expected that a variety of wands 26 will be used and may include flexible, regular and Frazier wand, and milled, flat tipped needles for use in urinary procedures or other small area needs. Complete with 12 feet of tubing for using the wands ad flat tipped needles at great distance from the machine. The apparatus 10 above described by way of example, is lightweight (15 lbs) and small in size (11′W×15″D×11′H), therefore very desirable for traveling veterinarians or quick movement from treatment room to treatment room by physicians.

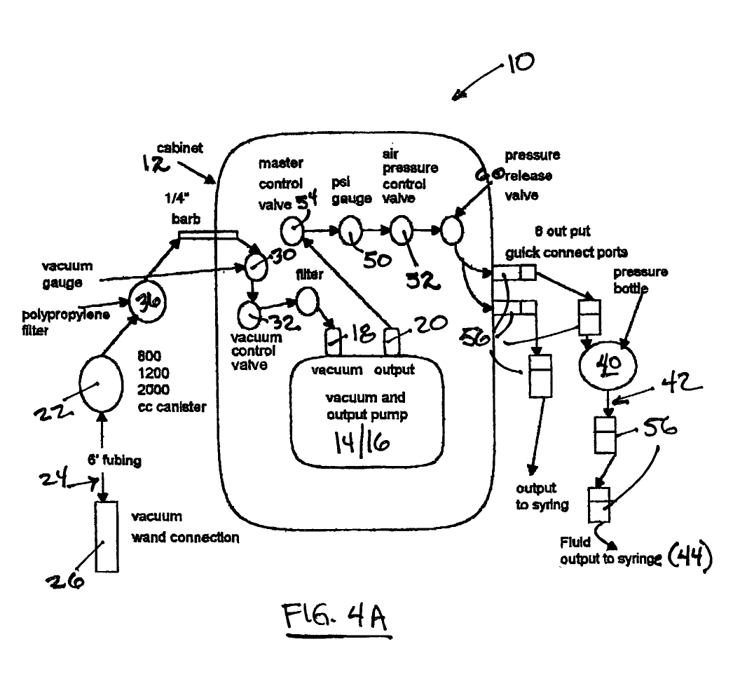

A Nebulizer attachment provides mist medicine and vapor treatments. The syringe used can operate up to 125 lbs of pressure, making fast cleaning of deep wounds a simple task. An 800 cc canister 22 is interchangeable with various sized canisters for different jobs. The canisters 22 include an overflow/shut-off valve 62 as illustrated with reference again to FIG. 3. The pressure bottle 40 generally used for spraying water, saline or medicine will hold 800 cc and can work at pressures up to 130 psi.

By way of further example, during operation of the apparatus 10, the separate vacuum and output controls allow an operator to adjust pressure settings to appropriate levels for the procedure performed. The external gauges provide reference at a glance. A fluid/compressed air delivery hand piece and a selection of plastic, autoclavable suction tips are used and a variety of commercially available stainless steel suction band pieces are compatible with the apparatus. The detachable tubing is autoclavable, making the apparatus ideal for use in sterile surgical conditions. As with any piece of medical equipment, patient compliance is important. The long tubing lengths used allow patient treatment at a sufficient distance from the unit to minimize the effect of mechanical noise. Patients will therefor tolerate many procedures without the need for sedation.

By way of further example, multiple functions are provided that can be divided into two operational categories: negative pressure (suction) and positive pressure (fluid/air output). The following describes procedures in each category. For each procedure listed there is information on apparatus settings, necessity of anesthesia/sedation and practice tips, by way of example only.

Negative Pressure (Suction) Procedures

1. Surgical Suction

A. Procedures/conditions: thoracotomy, laparotomy.

B. Anesthesia/sedation: By the nature of the procedure, anesthesia is mandatory.

C. Use autoclave sterilized band pieces and tubing. Begin procedure with maximum vacuum pressure.

D. Practice Tip: User preference may determine which suction tip to use. If tissue become repeatedly trapped into suction tip, decrease suction pressure.

2. Evacuation of fluid from body cavities

A. Procedures/conditions: hydrothorax” ascites.

B. Anesthesia/sedation: Most animals will allow fluid suction without sedation. If animal is fractious, sedation may be necessary, but the clinician must account for animal's compromised condition.

C. The time typically allotted for these procedures is important. Set the apparatus to maximum pressure to begin. If the suction is repeatedly blocked by tissue, the vacuum power can be reduced

D. Practice Tip: The patient should be placed in a comfortable position, but standing is ideal. In draining ascites, choose a ventral abdominal site, usually just lateral to the umbilicus. In draining a hydrothorax, a caudoventral intercostal site is used taking care to remain cranial to the diaphragm. Drainage sites should be clipped and surgically prepped.

To penetrate the cavity, use a large gauge (18 g or 20 g) needle or IV catheter. After entering the cavity, the needle/catheter should be directed parallel to the peritoneal/pleural surface to minimize risk of lacerating internal organs. The needle/catheter is connected to a three-way stopcock and the stopcock connected to the suction tubing. The needle/catheter may require periodic repositioning. either at the original penetration site or movement to a new penetration site. Vacuum pressure setting can be reduced if tissue is repeatedly occluding the needle/catheter.

When the suction canister requires emptying, turn the stopcock to close the needle/catheter. The vacuum pressure must be released in order to remove the canister lid; turn the apparatus off, turn the vacuum pressure control knob all the way the left disconnect the vacuum tubing from the canister lid and empty the canister. Once the lid is replaced and the vacuum tubing reattached, turn the apparatus on and turn the vacuum pressure control knob back to the right until the desired pressure setting is reached.

3. Evacuation of air from body cavities

A. Procedures/conditions: Closed pneumothorax GDV complex.

B. Anesthesia/sedation: Due to their physical nature when presented, these patients will generally allow treatment without sedation. If animal is fractious, sedation may be necessary, but the clinician must account for animal's compromised condition.

C. The time allotted for these procedures is important Set the apparatus to maximum pressure to begin with. If the suction is repeatedly blocked by tissue, the vacuum power can be reduced.

D. Practice Tip:

i. Pneumothorax patients should be placed in lateral recumbency and a site on the lateral rib cage of the “up” side of the thorax is clipped and surgically prepped.

To penetrate the cavity, use a large gauge (18 g or 20 g) needle or IV catheter. After entering the cavity at an intercostal space, the needle/catheter should be directed parallel to the pleural surface to minimize risk of lacerating lung tissue. The needle/catheter is connected to the suction tubing. The needle/catheter may require periodic repositioning, either at the original penetration site or movement to a new penetration site.

As patient's breathing stabilizes, gradually reduce the vacuum level setting. Discontinue suction when negative pressure is achieved. Due to mediastinal division of the thoracic cavity, it may be necessary to repeat the procedure on the opposite site of the chest. Post-procedural radiographs win help determine the success of the procedure.

ii. For GDV patients, the apparatus can be used to decompress the stomach when an orogastic tube cannot be passed Often after the stomach has been decompressed, a tube can be passed to allow gastric lavage.

Place the patient in right lateral recumbency. Clip and surgically prep a site on the left lateral abdomen over the level of the bloated stomach.

A large gauge (18 g or 20 g) needle is passed transdermally into the stomach. The vacuum tubing is attached to the needle. Using maximum vacuum pressure, the stomach is evacuated of gas.

If a gastric tube is successfully passed after decompression, the apparatus is used to suction lavage fluid from the stomach through the orogastric tube.

4. Pharyngeal suction

A. Procedures/conditions: nasal sinus flushing, oropharyngeal surgery.

B. Anesthesia/sedation: By the nature of the procedure, anesthesia with intubation is mandatory.

C. Low to mid vacuum levels (20 to 40) are adequate for suction without traumatizing pharyngeal tissues.

D. Practice Tip: Position the patient in either dorsal or ventral recumbency to allow easiest exposure to pharynx. A spring-loaded mouth gag is helpful to keep mouth open. Choose the suction hand piece that best suits operator's preference. See Nasal Sinus Flushing below for additional tips.

Positive Pressure (Fluid/Compressed Air Delivery) Functions

Important Note: If the apparatus must be turned off during a positive pressure procedure, the established pressure must be released from the compressor before the unit can be turned back on. Open the positive pressure knob all the way to the left until the positive pressure gauge returns to zero. After restarting the apparatus, turn the positive pressure knob to the right until the desired output pressure is reestablished.

1. Ear Flushing

A. Procedures/conditions: Otitis Externa ear mite infestation.

B. Anesthesia/sedation: Most patients will allow flushing of ears without sedation. Fractious or painful animals may be sedated if necessary.

C. Fill the fluid delivery bottle ⅔ full with warm water or saline. An antiseptic solution like chlorhexidine may be diluted into flush solution. Start with an output pressure of 40. Depending on the size of the patient and the amount of debris to be removed, the pressure may be increased or decreased. Test the pressure by spraying flush solution against the palm of your hand before flushing ear.

D. Practice Tip: Position the patient over a wet sink, or have an assistant hold a bowl below the patient's ear. For ears with excessive discharge, apply a surfactant solution (e.g. ClearRX) before flushing.

Insert the tip of the positive pressure hand piece into the ear canal. Infuse a steady stream of flush solution into the ear. Use caution to not depress the air delivery button on the positive pressure hand piece. Most patients do not like the induction of bubbles into the ear canal.

Check ear canal with an otoscope to assess success of flushing. Repeat procedure in opposite ear if necessary.

2. Wound/abscess flushing

A. Procedure/conditions: Any abrasions, lacerations, abscesses

B. Anesthesia/sedation: If patient is painful or fractious, sedation is advisable.

C. Fill the fluid delivery bottle ⅔ full with warm water or saline. An antiseptic solution like chlorhexidine may be diluted into flush solution. Start with an output pressure of 40. Depending on the size of the patient and the amount of debris to be removed, the pressure may be increased or decreased.

D. Practice Tip: Place the patient over a wet sink or on a thick towel. Clip and clean the skin around wound For abscesses, lance the area using a scalpel blade and manually express the purulent discharge. Flush the wound using the fluid output button on the positive pressure hand piece until the wound is clean.

3. Wound Hydrotherapy

A. Procedure/conditions: repetitive cleaning of open wounds and degloving injuries.

B. Anesthesia/sedation: Most patients win tolerate daily cleaning without sedation.

C. Fill the fluid delivery bottle ⅔ full with warm water or saline. An antiseptic solution like chlorbexidine may be diluted into flush solution. Start with an output pressure of 40. Depending on the size of the patient and the amount of debris to be removed the pressure may be increased or decreased.

D. Practice Tip: Place the patient over a wet sink or on a thick towel. Flush the wound using the fluid output button on the positive pressure hand piece until the wound is clean. Daily hydrotherapy win help stimulate granulation tissue. After cleaning, topical medications, conventional bandages, or wet to dry bandages may be applied.

4. Body Cavity lavage

A. Procedure/conditions: cleaning a contaminated body cavity during laparotomy or thoracotomy.

B. Anesthesia/sedation: By the nature of the procedure, anesthesia is mandatory.

C. Fill the fluid delivery bottle ⅔ full with warm water or saline. Set the output pressure to maximum.

D. Practice Tip: Lavage body cavity to remove contamination and debris. Evacuate the cavity using the suction capabilities as above.

5. Sinus Flushing

A. Procedures/conditions: Chronic sinusitis, infections, and nasal foreign bodies.

B. Anesthesia/sedation: by the nature of the procedure, anesthesia with intubation is mandatory.

C. This procedure will utilize both the positive and negative pressure applications of the apparatus. See Pharyngeal suction above for negative pressure settings. Fill the fluid delivery bottle ⅔ full with warm water or saline. An output pressure of 40 to 60 is applicable, depending on patient size.

D. Practice Tip: Place the anesthetized intubated patient in dorsal recumbency. A spring-loaded mouth gag is helpful to keep the mouth open. Insert the tip of the positive pressure hand piece into one nostril and depress the fluid delivery button on the hand piece. As flush solution enters the pharyngeal cavity, it is removed with suction.

6. Nebulization for inhaled medications

A. Procedure/conditions: Respiratory infections or conditions benefiting from humidified air.

B. Anesthesia/sedation: Not necessary.

C. Attach the nebulizer unit to the positive pressure hand piece. Saline, with or without medication is placed in the nebulizing chamber. Depress the air delivery button on the positive pressure band piece.

D. Practice Tip: Hold the nebulizer near the patient's nose and mouth to deliver humidified air and nebulized medications. This method can be used to administer antibiotics,. brochodilators. and mucolytics via inhalation.

7. Instrument cleaning

A. Procedure/conditions: Soiled instruments and clipper blades.

B. Anesthesia/sedation: N/A

C. Set positive pressure to maximum.

D. Practice Tip: Fluid and air delivery can be used to remove debris from clipper blades and surgical instruments.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that modifications and alternate embodiments are intended to be included within the scope of the appended claims.

That which is claimed is:

1. A portable apparatus for rapidly irrigating and aspirating living body portions to thus avoid anesthetizing thereof, the apparatus comprising:
a housing dimensioned for manual placement at a preselected location;
a piston driven motor carried by the housing;
a vacuum pump carried by the housing and operable with the piston driven motor, the vacuum pump having an input port receiving air flow therein for providing a vacuum to vacuum levels exceeding approximately twenty seven inches of mercury, and an output port for expelling air therethrough under pressure to pressure levels exceeding approximately one hundred an twenty pounds per square inch;
a canister in fluid flow communication with the input port;
an aspirator wand in fluid flow communication with the canister for depositing fluid therein as a result of the vacuum;
vacuum level control means operable between the input port and the canister for controlling the vacuum developed at the canister and thus at the aspirator wand for receiving body fluids at a preselected flow rate, wherein the vacuum level control means comprise a gauge for displaying the vacuum level and a manually actuated valve for controlling the vacuum level;
a ceramic filter carried by the housing, the ceramic filter in fluid communication between the input port and the vacuum control means for filtering flow from the canister to the input port and preventing debris form entering the vacuum pump;
an inline filter operable with the canister for filtering flow therefrom;
a pressure bottle in fluid flow communication with the output port;
a syringe in fluid flow communication with the output port for delivering air under pressure therefrom, the syringe further being in fluid flow communication with the pressure bottle for delivering air under pressure mixed with fluid from the pressure bottle;
flow control means operable with the syringe for controlling fluid flow from at least one of the output port and the pressure bottle;
pressure level control means operable with the output port for controlling the air pressure delivered therefrom, and thus to the syringe and pressure bottle, wherein the pressure level control means comprise a gauge for displaying the pressure level and a manually actuated valve for controlling the pressure level;
a second pressure valve operable with the output port for limiting pressure delivered therefrom; and
a plurality of quick-connect connections positioned inline between at least one of the output port and pressure control means, the pressure control means and the pressure bottle, the pressure bottle and the syringe, and the pressure control means and the syringe.
